(19) United States
(12) Patent Application Publication
(10) Pub. No.: US 2023/0365489 A1
(43) Pub. Date: Nov. 16, 2023

(54) PROCESS FOR THE PURIFICATION OF PHENYLENEDIAMINES

(71) Applicant: Jianjun SUI, Singapore (SG)

(72) Inventor: Jianjun SUI, Singapore (SG)

(21) Appl. No.: 18/030,004

(22) PCT Filed: Oct. 3, 2020
(86) PCT No.: PCT/SG2020/050559

§ 371 Date: Apr. 3, 2023

(51) Int. Cl. C07C 211/51; B01D 3/14; B01D 9/00

(52) U.S. Cl. CPC C07C 211/51; B01D 3/141; B01D 9/004

(57) ABSTRACT

The present invention relates to process for separating p-phenylenediamine from a mixture containing o-phenylenediamine, aminobiphenyls and diphenylamine by means of distillation coupled with crystallization, wherein the distillation system consists of two divided wall columns and the crystallization comprises at least one stage suspension-based melt crystallization. A liquid fraction enriched in p-phenylenediamine obtained as the side-draw of the first divided wall column is sent to the subsequent suspension-based melt crystallization to produce substantially pure p-phenylenediamine. The overhead product of the first divided wall column is fed to the second divided wall column to produce highly pure o-phenylenediamine withdrawn from the side-draw of the second divided wall column.

### BACKGROUND OF THE INVENTION

This present invention relates to a process for the separation of p-phenylenediamine from a mixture containing o-phenylenediamine, aminobiphenyl, diphenylamine, aniline, biphenyl and tars by means of distillation coupled with crystallization. More specifically, the invention relates to a process for economically preparing substantially pure p-phenylenediamine from said mixture by a combination of two divided wall columns and at least one stage suspension-based melt crystallization.

P-phenylenediamine is particularly useful as important intermediates in the production of aramid textile fibers such as Kevlar and Twaron, hair dyes and antiozonants for rubber products. The two most important commercial routes for synthesizing p-phenylenediamine are the catalytic hydrogenation of p-nitroaniline and aniline diazotization. In the p-nitroaniline route, chlorobenzene reacts with nitric acid to obtain p-nitrochlorobenzene, Which is aminated with ammonia to p-nitroaniline, followed by hydrogenation of p-nitroaniline to produce p-phenylenediamine. The second route involves the diazotization of aniline using nitrogen oxide, reaction with an excess of aniline to form 1,3-diphenyltriazene, and rearrangement of 1,3-diphenyltriazene to p-aminoazobenzene, which is then catalytically cleaved and hydrogenated to give p-phenylenediamine and aniline, see e.g. U.S. Pat. No. 4,020,052 and U.S. Pat. No. 4,279,815. Most of the aniline is recovered and recycled.

A disadvantage of p-nitroaniline route is the production of a quite high amount of o-nitrochlorobenzene during nitration of chlorobenzene, which results in the yield of p-phenylenediamine reduced. On the other hand, the aniline diazotization route can produce p-phenylenediamine in a relatively high yield under mild reaction conditions as it is based only on aniline. Therefore, the aniline diazotization route to produce p-phenylenediamine is widely carried out on a commercial scale.

However, during the diazotization of aniline with a nitrogen oxide-containing gas and the rearrangement of the 1,3-diphenyltriazene, side-reactions leading to the formation of polynuclear (chiefly binuclear) by-products such as diphenylamine and aminobiphenyls, particularly 2-aminobiphenyl, may occur. Also, yield losses to the undesired o-aminoazobenzene isomer may result. As is described in British Pat. No. 1,430,366, several % by weight of o-phenylenediamine as a by-product is unavoidably contained in the final reaction product. The said final reaction product which is essentially free of aniline can contain, for example, 70 to 85% by weight of p-phenylenediamine, 4 to 15% by weight of o-phenylenediamine, 0 to 15% by weight of aminobiphenyls, 0 to 8% by weight of diphenylamine, 0 to 2% by weight of aniline, 0 to 2% by weight of biphenyl and 0 to 2% by weight of tars.

Various processes have been proposed for preparing highly pure p-phenylenediamine from a multi-component mixture. U.S. Pat. No. 4,191,708, for example, describes a process, wherein o-aminoazobenzene produced as a by-product in the step of preparing p-aminoazobenzene is separated and removed from the reaction product containing p-ammoazobenzene and o-aminoazobenzen.e by crystallization from a solvent before p-aminoazobenzene is reduced to produce p-phenylenediamine. A subsequent distillation at reduced pressure gives p-phenylenediamine having a purity of 99.8% by weight. One disadvantage of the process is its elevated energy consumption and equipment investment in the step of solution crystallization, which involves several operation units including crystallization, centrifugation, drying, and solvent recovery by distillation.

Recently there is a growing demand for ultrapure p-phenylenediamine having a purity of 99.99% by weight for use in the polymer industry. Melt crystallization can meet industry's need for a highly selective separation process and has the added advantage that it is energy saving and ecologically sound since no solvent is added. Melt crystallization can be subdivided into layer crystallization and suspension-based melt crystallization based on their different ways of obtaining the liquid crystal product. Of particular interest here is a separation technique comprising suspension-based melt crystallization, which is more energy-efficient and has a better separation efficiency than layer crystallization.

To produce p-phenylenediamine with a desired purity from the suspension-based melt crystallization the said reaction product stream is typically fed to a distillation system to obtain a liquid fraction enriched in p-phenylenediamine serving as the teed for crystallization. Specifically, the lights such as residual aniline and biphenyl which have a boiling point lower than o-phenylenediamine are distilled out from the top of the first distillation column and the bottoms product containing phenylenediatnines, aminobiphenyls and diphenylamine are introduced to the second distillation column to obtain highly pure o-phenylenediamine as the overhead product of the second distillation column. The bottoms product of the second distillation column is fed to the third distillation column to obtain a liquid fraction enriched in p-phenylenediamine from the top serving as the feed to the subsequent crystallization, and aminobiphenyls, diphenylamine and other heavies are removed from the bottom of the third distillation column. Such an arrangement of the three distillation columns is in general referred to as a conventional column sequence.

While such an above-mentioned three-column distillation sequence allows to obtain a liquid fraction enriched in p-phenylenediamine, one disadvantage is its elevated energy requirement. In conventional distillation columns, the feed stream is conventionally fractional into two product streams: an overhead product and a bottoms product. Any further separations which are required may, for example be performed by subjecting either the bottoms product stream or the overhead product stream to another distillation column similar to the first. The operating costs of such a three-column distillation sequence are correspondingly high. In the case of three-column distillation sequence for the purpose of obtaining a concentrated p-phenylenediamine liquid fraction, each of the three columns has to be supplied with the thermal energy required to perform the evaporation of the liquid and the separation of the mixture.

On the other hand, the investment costs for the three-column distillation sequence are high. This is not only due to the fact that investment has to be made for the three distillation columns, but expenses are also necessary for the equipment associated therewith, for example condensers, drums, reboilers and pumps.

In addition, it has been recognized that the extent of forming tars is increasing with the increase of the exposure of p-phenylenediamine to elevated temperatures at the presence of in-leakage air over multi-distillation stages, which distillation is generally operated at a reduced pressure. In the case of three-column distillation sequence the liquid fraction enriched in p-phenylenediamine is only obtained in the last distillation column, which indicates that the residence time of p-phenylenediamine in the conventional column sequence is relatively long, resulting in increased unfavorable side-reactions of p-phenylenediamine to form tars and leading to a loss of product of p-phenylenediamine.

Furthermore, regarding the last column in the conventional sequence it is noticed that the melting point of p-phenylenediamine is 140° C., which should limit the top vapors of the column to the condenser located directly downstream of the column, to a temperature of at least 160° C., i.e. a safety margin of 20° C. is required so as to prevent any solidification of the enriched p-phenylenediamine on the surface of the tubes in the condenser. The setting of the top vapor temperature as 160° C. correlates with a pressure of 35 mbar at the top of the column, which indicates that it should operate at a top pressure higher than 35 mbar.

### SUMMARY OF THE INVENTION

It is therefore the object of this invention to develop a process based on two divided wall columns arid suspension-based melt crystallization, by means of which p-phenylenediamine can be separated. from mixtures which containaminobiphenyl, o-phenylenediamine, diphenylamine, aniline, biphenyl and tars. The first divided wall column required for the purpose can produce enriched p-phenylenediamine from the outlet of its side-draw, so that the residence time of p-phenylenediamine in the distillation was greatly reduced and the top pressure of the column can be reduced below 35 mbar, all of which contribute to the less formation of tars. The invention also pertains to produce high purity o-phenylenediamine from the side-draw of the second divided wall column. Another advantage is that the proposed divided wall columns coupled with the suspension-based melt crystallization are to save equipment investment costs and energy input compared with the above-mentioned conventional distillation sequence.

### BRIEF DESCRIPTION OF THE DRAWING

FIG. 1 is a schematic diagram of a preferred phenylenediamine purification system utilizing a combination of two divided wall columns and one stage suspension-based melt crystallization.

### DETAILED DESCRIPTION OF THE PREFERRED EMBODIMENTS OF THE INVENTION

With regard to the conventional three-column distillationsequence, the liquid fraction enriched in p-phenylenediamine is drawn off from the top of the third distillation column. In order to minimize the amount of tars formed in distillation stages, an improved process is provided to obtain a concentrated p-phenylenediamine fraction from the side-draw of the first distillation column as the feed to a subsequent suspension-based melt crystallization, which first column is a divided wall column. Thus, the residence time of p-phenylenediamine is greatly reduced, the exposure to the elevated temperatures and in-leakage air is less and the yield of p-phenylenediamine is increased. The lights including aniline and biphenyl, phenylenediamine and some parts of p-phenylenediamine are distilled off from the top of the first divided wall column and fed to a subsequent second divided wall column, wherein highly pure o-phenylenediamine is removed from the side-draw of the second divided wall column.

In accordance wwith the present invention, the three-column distillation sequence is replaced by two divided wall columns. The expenditure on plant and equipment and the space required for the installation of the distillation columns are significantly decreased.

Moreover, it is observed that utilizing of the two divided wall columns in replace of the three conventional distillation columns has advantages that not only the energy consumption is reduced in comparison to the process known in the prior art, but that the liquid fraction enriched in p-phenylenediamine with at least the same purity is obtained, meaning the separation efficiency of the proposed process is comparable with the above-mentioned three-column distillation sequence.

In accordance with the usual definition of the term “divided wall column”, the divided wall column preferably comprises:
a divided wall provided vertically inside the column shell, defining a divided oval section between an upper undivided section as a rectifying zone and a lower undivided section as a stripping zone;
a divided oval section located between the rectifying zone and the stripping zone having a vertical dividing wall dividing the inner space of the divided wall section into a pre-fractionation zone at one side of the divided wall and a main fractionation zone at the other side of the divided wall;
an inlet for the feed stream in the pre-fractionation zone, a side-draw outlet in the main fractionation zone, an overhead product stream drawn off from the rectification zone, and a bottoms product stream removed from the stripping zone.

The distillation in each of the divided wall columns is preferably carried out at low temperatures and reduced pressures. Lower temperatures minimize the occurrence of side-reactions of phenylenediamine, which can lead to formation of tars and result in a loss of product. The pressures at the top of each divided wall column are preferably in the ranges of 20 to 120 mbar The pressures at the bottom of each divided wall column are preferably in the ranges of 40 to 130 mbar.

The present invention is not particularly limited with regard to the type of mass transfer elements installed in the divided wall columns. Good results are obtained by using suitable mass transfer elements selected from the group consisting of random packings, structured packings and any combinations thereof. It is however, structured packings are particularly suitable as mass transfer elements with the advantages of reducing the liquid hold-up in the column and resulting in a lower pressure drop over the column. It is preferred that the structured packings have a specific surface area in the range of 125 to 750 m²/m³, and more preferably in the range of 250 to 500 m² m³.

The length of the divided wall in the divided wall section depends on the process conditions and on the mass transfer elements used. In each of the divided wall columns of the present invention, the length of each divided wall is approximately ⅔ of the total length of the mass transfer elements portion installed in the corresponding divided wall column. It is preferred that the total mass transfer elements portion in each divided wall column has a length between 10,000 and 35,000 mm, and more preferably between 15,000 and 25,000 mm,

In each of the divided wall columns of the present invention, the divided wail section is partitioned by the divided wall into a pre-fractionation zone and a main fractionation zone, which each has a different volume, i.e. a different cross-sectional area for each zone. Different processes may be optimized by appropriate selection of the partial cross-sections of the two zones.

Vapor flow from the stripping zone is divided in the pre-fractionation zone and the main fractionation zone in accordance with the cross-sectional area of each zone. The partial cross-sectional areas are set in such a manner that the pressures at the inlet and outlet regions of the pre-fractionation zone are respectively identical with those at the inlet and outlet regions of the main fractionation zone, which means the total pressure drop over the packings within the pre-fractionation zone is the same as that for the packings within the main fractionation zone.

In accordance with the present invention, each of the divided wall columns is equipped with at least one reboiler to generate the energy required for the evaporation and at least one condenser to condense the overhead vapor stream. The reboiler can be of any of the types commonly found in the chemical industry, including, but not limited to, falling-film evaporators, forced circulation evaporators, thermosiphon evaporators and etc. However, due to its particularly reduced liquid hold-up, a falling film evaporator is preferred to minimize the residence time of phenylenediamines in the reboiler and therefore reduce any unfavorable side-reactions. The condenser can be of any of the types commonly used in the chemical industry including co-current and counter-current condensers.

In the process of the first divided wall column, a concentration gradient is established with the lower-boiling point components of having a lower boiling point than p-phenylenediamine being enriched in the rectifying zone, and the higher boiling point components of having a higher boiling point than p-phenylenediamine being enriched in the stripping zone. The overhead vapor stream at the top of the divided wall column is condensed by means of a condenser to obtain a condensate streatm Vapors that have not been condensed in the condenser may be fed to an additional condenser for further condensation. A portion of said condensate stream is refluxed into the column while the other portion is removed as the overhead product stream containing a major portion of o-phenylenediamine and p-phenylenediamine and a small portion of lights including aniline and biphenyl. The overhead product stream from the divided wall column is fed to the pre-fractionation zone of the second divided wall column. The liquid bottom stream concentrated in the stripping zone is drawn off from the bottom of the divided wall column and subsequently divided into a bottoms product stream comprising a major portion of aminobiphenyl and diphenylarnine and a small portion of p-phenylenediamine and tars, and a recirculation stream to be reboiled in the falling film evaporator. The increase in the amount of tars in the bottoms product stream is observed due to the side-reactions occurring under the relatively high temperature at the bottom of the divided wall column. The concentrated p-phenylenediamine product stream as the side-draw is withdrawn from the main fractionation zone of the divided wall column, containing a major portion of p-phenylenediamine and a minor portion of o-phenylenediamine, aminobiphenyl and diphenylaniine. In order to obtain an ultrapure p-phenylenediamine the concentrated p-phenylenediamine stream is subjected to a further purification step, which step comprises one stage suspension-based melt crystallization.

In the process of the second divided wall column, a concentration gradient is established with the lower-boiling point components of having a lower boiling point than o-phenylenediamine being enriched in the rectifying zone, and the higher-boiling point components of having a higher boiling point than o-phenylenediamine being enriched in the stripping zone. The overhead vapor stream at the top of the divided wall column is condensed by means of a condenser to obtain a condensate stream. Vapors that have not been condensed in the condenser may be fed to an additional condenser for further condensation. A portion of said condensate stream is refluxed into the column while the other portion is removed as the overhead product stream containing a major portion of aniline and biphenyl and a small portion of o-phenylenediamine. The liquid bottom stream concentrated in the stripping zone is drawn off from the bottom of the divided wall column and subsequently divided into a bottoms product stream comprising a major portion of p-phenylenediamine and a small portion of o-phenylenediamine and tars, and a recirculation stream to be reboiled in the falling film evaporator. In order to improve the overall recovery of p-phenylenediamine, the bottoms product stream is preferably recycled to the inlet of the first divided wall column. The o-phenylenediamine product stream as the side-draw is withdrawn from the main fractionation zone of the divided wall column, consisting essentially of o-phenylenediainine.

The proposed crystallization process comprises one stage continuous suspension-based melt crystallization for the purification of the concentrated p-phenylenediamine product stream. The suspension-based melt crystallization system consists of a single scraped surface drum crystallizer and at least one wash column, providing efficient separation of the p-phenylenediamine crystals from the impurity enriched mother liquor and discharging ultrapure liquid p-phenylenediamine product.

The concentrated p-phenylenediamine product stream enters the scraped surface drum crystallizer where it is cooled by circulating heat transfer fluid in the outer jacket to create a supersaturated film of solution at the inner wall of the crystallizer. The inner wall of the crystallizer is scraped to prevent encrustation from fouling the cooling surface and therefore maintains efficient continuous operation. The sub-cooled liquid is quickly mixed with the crystallizer volume allowing the supersaturation to be released over the bulk slurry where a stable and controlled crystal growth environment is generated. The resulting slurry is fed to the wash column for complete separation of the pure p-phenylenediamine crystals from the impurity rich mother liquor.

In the wash column, most of the impurity rich mother liquor is removed from the p-phenylenediamine crystals by filtration or other means and the crystals essentially free of mot her liquor form a compacted crystal bed. While the crystals are completely pure p-phenylenediamine the residual mother liquor will form a thin film that adheres to the outside surface of the crystals. Part of the substantially pure phenylenediamine crystals are cut off from the crystal bed and melted using steam or other su itable heat source. A small amount of the melted p-phenylenediamine crystals is pressed through the crystal bed to wash out adhering mother liquor while most of the melted phenylenediamine is discharged from the system as the final p-phenylenediamine product via an automated control valve. The purge stream from the wash column is recycled to the feed stream to the first divided wall column.

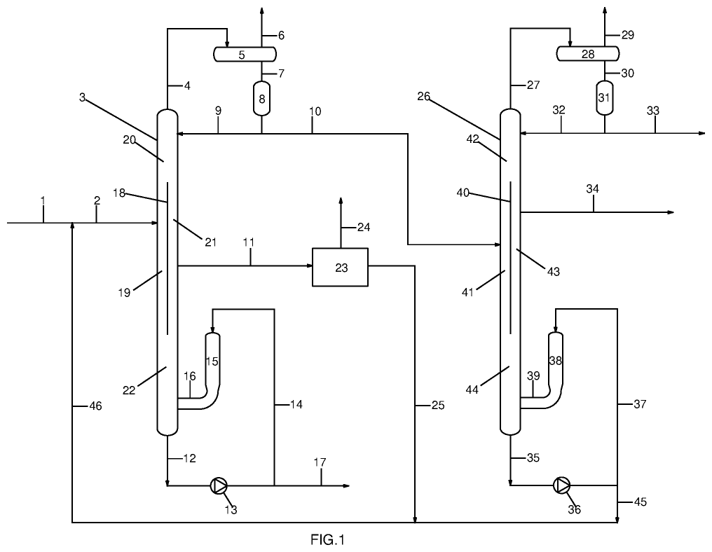

FIG. 1 schematically shows a preferred p-phenylenediamine purification system in accordance with the present invention, which system consists of two divided wall columns and one stage suspension-based melt crystallization.

The first divided wall column comprises a column shell 3, a condenser 5, a condensate drwn 8, a circulation pump 13, a falling film reboiler 15, a substantially fluid tight divided wall 18 extending vertically through the middle part of the column shell 3. The inner space of the column shell 3 is divided by the divided wall 18 into four distinct zones, i.e. a pre-fractionation zone 19 at one side of the divided wall 18, a rectifying zone 20 above the divided wall 18, a main fractionation zone 21 at the other side of the divided wall 18, and a stripping zone 22 below the divided wall 18, in which column the pre-fractionation zone 19 and the main fractionation zone 21 form the divided wall section. The vapors generated at the bottom of the divided wall column flow upwards through the stripping zone 22 and divide into the pre-fractionation zone 19 and the main fractionation zone 21, counter-currently contacting the liquids flowing downwards from rectifying zone 20, effective for a mass transfer. A multi-component feed stream 2 is then separated by the mass transfer within the four operating zones into three product streams, i.e. an overhead product stream 10, a bottoms product stream 17 and a side-draw product stream 11.

The second divided wall column comprises a column shell 26, a condenser 28, a condensate drum 31, a circulation pump 36, a falling film reboiler 38, a substantially fluid tight divided wall 40 extending vertically through the middle part of the column shell 26. The inner space of the column shell 26 is divided by the divided wall 40 into four distinct zones, i.e. a pre-fractionation zone 41 at one side of the divided wall 40, a rectifying zone 42 above the divided wall 40, a main fractionation zone 43 at the other side of the divided wall 40, and a stripping zone 44 below the divided wall 40, in which column the pre-fractionation zone 41 and the main fractionation zone 43 form the divided wall section. The vapors generated at the bottom of the divided wall column flow upwards through the stripping zone 44 and divide into the pre-fractionation zone 41 and the main fractionation zone 43, counter-currently contacting the liquids flowing downwards from rectifying zone 42, effective for a mass transfer.: multi-component feed stream 10 is then separated by the mass transfer within the four operating zones into three product streams, i.e. an overhead product stream 33, a bottoms product stream 45 and a side-draw product stream 34.

The said reaction product stream 1 combining with stream 46 is continuously fed through feed stream 2 into the pre-fractionation zone 19 of the first divided wall column. The lower-boiling components of having a lower boiling point than p-phenylenediamine are concentrated during the distillation in the rectifying zone 20, and are drawn off through stream 4, which is subsequently condensed in the condenser 5, The condensates flow to the condensate drum 8 through stream 7 and then divide into an overhead product stream 10 distilled out from the top and into a reflux stream 9, which is fed back to the rectifying zone 20. The uncondensed vapors are removed through stream 6. The higher-boiling components of having a higher boiling point than p-phenylenediamine are concentrated in the stripping zone 22 and drawn off as a bottom stream 12. The bottom stream 12 is subsequently divided into a bottoms product stream 17 withdrawn from the bottom of the column, and a recirculation stream 14 to he reboiled in the falling film evaporator 15 and then fed back to the stripping zone 22 through stream 16. A side-draw product of the concentrated p-phenylenediamine is withdrawn through stream 11 from the main fractionation zone 21 and fed to the suspension-based melt crystallization system 23 for further purification. The ultrapure p-phenylenediamine is obtained as stream 24 and the purge stream 25 discharged from the crystallization system 23 is recycled to stream 46.

The overhead product stream 10 from the first divided wall column as the feed stream is continuously fed to the pre-fractionation zone 41 of the second divided wall column. The lower-boiling components of having a lower boiling point than o-phenylenediamine are concentrated during the distillation in the rectifying zone 42, and are drawn off through stream 27, which is subsequently condensed in the condenser 28. The condensates flow to the condensate drum 31 through stream 30 and then divide into an overhead product stream 33 distilled off from the top and into a reflux stream 32, which is recycled to the rectifying zone 42, The uncondensed vapors are removed through stream 29. The higher-boiling components of having a higher boiling point than o-phenylenediamine are concentrated in the stripping zone 44 and drawn off as a bottom stream 35, The bottom stream 35 is subsequently divided. into a bottoms product stream 45 withdrawn from the bottom of the column, and a recirculation stream 37 to be rehoiled in the falling film evaporator 38 and then fed back to the stripping zone 44 through stream 39. A side-draw product of the highly pure o-phenylenediamine is withdrawn through stream 34 from the main fractionation zone 43. The bottoms product stream 45 is recycled to stream 46.

in accordance with the present invention, the use of two divided-wall columns with one stage suspension-based melt crystallization to obtain the substantially pure p-phenylenediamine from said reaction product makes it possible to save one distillation column in comparison with the above-mentioned three-column distillation sequence process. It has advantages that not only the energy consumption and equipment expenditure are reduced, but also the residence time of phenyle ediamines is lessened, resulting in less tars formed due to its reduced exposure to the elevated temperatures and in-leakage air.

Subsequently, the present invention is illustrated in more details below with reference to the drawing and the examples.

### EXAMPLES

### Example 1

A distillation in the first divided wall column according to an embodiment of the invention as shown in FIG. I was performed. Structured packings with a specific surface area of 345 m²/m³ were used as mass exchange elements in e divided wall column. 53% by weight of the liquid from the rectifying zone 20 was introduced to the pre-fractionating zone 19 and 47% by weight o f the liquid from the rectifying zone 20 to the main fractionating zone 21. The rectifying zone 20 had 11 theoretical stages and the stripping zone 22 had 9 theoretical stages. The pre-fractionation zone 19 had 11 theoretical stages above and 25 theoretical stages below the feeding point of stream 2 into the pre-fractionation zone 19. The main fractionation zone 21 had 12 theoretical stages above and 24 theoretical stages below the withdrawal point of the side-draw product stream 11 from the main fractionating zone 21. The overhead pressure was 55 mbar. The reflux ratio at the withdrawal point of the overhead product streams 10 was 21:1. The pressure and temperature at the bottom of the divided wall column were 70 mbar and 197° C., respectively.

A distillation in the second divided wall column according to an embodiment of the invention as shown in FIG. 1 was performed. Structured packings with a specific surface area of 495 m²/m³ were used as mass exchange elements in the divided wall column. 42% by weight of the liquid from the rectifying zone 42 was introduced to the pre-fractionating zone 41 and 58% by weight of the liquid from the rectifying zone 42 to the main fractionating zone 43. The rectifying zone 42 had 5 theoretical stages and the stripping zone 44 had 25 theoretical stages. The pre-fractionation zone 41 had 26 theoretical stages above and 14 theoretical stages below the feeding point of stream 10 into the pre-fractionation zone 41. The main fractionation zone 43 had 16 theoretical stages above and 24 theoretical stages below the withdrawal point of the side-draw product stream 34 from the main fractionating zone 43, The overhead pressure was 55 mbar. The reflux ratio at the withdrawal point of the overhead product stream 33 was 60:1. The pressure and temperature at the bottom of the divided wall column were 70 mbar and 181° C., respectively.

4000 kg/h of the reaction product stream 1 composed of 70.37% by weight of p-phenylenediamine, 7.24% by weight of o-phenylenediatnine, 13.89% by weight of 2-aminobiphenyl, 6.51% by weight of diphenylamine, 0.81% by weight of aniline, 0.51% by weight of biphenyl and 0.66% by weight of tars, combined with stream 46 and was continuously fed to the pre-fractionation zone 19 of the first divided wall column through feed stream 2. The overhead product stream 10 of the first divided wall column was fed to the pre-fractionation zone 41 of the second divided wall column for further purification and the bottoms product stream 17 of the first divided wall column was removed, The liquid side-draw product stream 11 enriched in p-phenylenediamine was subjected subsequently to the suspension-based melt crystallization system 23 to obtain substantially pure p-phenylenediamine product having a purity of at least 99.99% by weight of p-phenylenedianilne through stream 24. The total energy consumption was as low as 2.9 MAY for the two divided wall columns. The compositions of different streams were tabulated in the following table.

| | | First divided wall column | | | |
|---|---|---|---|---|---|
| | | | | | Liquid |
| | | | Overhead | Bottoms | side-draw |
| | | Feed | product | product | product |
| | | stream | stream | stream | stream |
| P-phenylenediamine | wt. % | 77.31 | 40.16 | 6.35 | 98.70 |
| O-phenylenediamine | wt. % | 6.19 | 52.52 | 0 | 0.81 |
| 2-aminobiphenyl | wt. % | 10.30 | 0 | 58.97 | 0.39 |
| Diphenylamine | wt. % | 4.76 | 0 | 27.64 | 0.10 |
| Aniline | wt. % | 0.58 | 3.88 | 0 | 0 |
| Biphenyl | wt. % | 0.37 | 3.44 | 0 | 0 |
| Tars | wt. % | 0.48 | 0 | 7.03 | 0 |

| | | Second divided wall column | | | |
|---|---|---|---|---|---|
| | | | | | Liquid |
| | | | Overhead | Bottoms | side-draw |
| | | Feed | product | product | product |
| | | stream | stream | stream | stream |
| P-phenylenediamine | wt. % | 40.16 | 0 | 91.07 | 0.10 |
| O-phenylenediamine | wt. % | 52.52 | 20.86 | 8.66 | 99.71 |
| 2-aminobiphenyl | wt. % | 0 | 0 | 0 | 0 |
| Diphenylamine | wt. % | 0 | 0 | 0 | 0 |
| Aniline | wt. % | 3.88 | 36.51 | 0 | 0 |
| Biphenyl | wt. % | 3.44 | 42.62 | 0 | 0.18 |
| Tars | wt. % | 0 | 0 | 0.27 | 0 |

| | | Suspension-based melt crystallization | | |
|---|---|---|---|---|
| | | Feed | Product | Purge |
| | | stream | stream | stream |
| P-pbenylenediamine | wt. % | 98.70 | 99.99 | 96.00 |
| O-phenylenediamine | wt. % | 0.81 | 0.01 | 2.49 |
| 2-aminobipheny] | wt. % | 0.39 | 0 | 1.21 |
| Diphenylamine | wt. % | 0.10 | 0 | 0.31 |
| Aniline | wt. % | 0 | 0 | 0 |
| Biphenyl | wt. % | 0 | 0 | 0 |
| Tars | wt. % | 0 | 0 | 0 |

### Comparative Example 1

A distillation in the aforementioned three-column distillation sequence was performed to obtain the concentrated p-phenylenediamine product having a purity of 98.65% by weight of p-phenylenediamine in the third distillation column and the highly pure o-phenylenediamine product having a purity of 99.66% by weight of o-phenylenediamine in the second distillation column, respectively. The concentrated p-phenylenediamine product from the third distillation column was subjected subsequently to a suspension-based melt crystallization system to obtain substantially pure p-phenylenediamine. The total amount of structured packings installed in the three distillation columns were similar as those for the two divided wall columns described in Example 1. The total energy consumption for the three distillation columns was 4.6 MW.

### Example 2

A distillation in the first divided wall column according to an embodiment of the invention as shown in FIG. 1 was performed. Structured packings with a specific surface area of 345 m²/m³ were used as mass exchange elements in the divided wall column. 57% by weight of the liquid from the rectifying zone 20 was introduced to the pre-fractionating zone 19 and 43% by weight of the liquid from the rectifying zone 20 to the main fractionating zone 21. The rectifying zone 20 had 11 theoretical stages and the stripping zone 22 had 9 theoretical stages. The pre-fractionation zone 19 had 11 theoretical stages above and 25 theoretical stages below the feeding point of stream 2 into the pre-fractionation zone 19. The main fractionation zone 21 had 12 theoretical stages above and 24 theoretical stages below the withdrawal point of the side-draw product stream 11 from the main fractionating zone 21. The overhead pressure was 65 mbar. The reflux ratio at the withdrawal point of the overhead product stream 10 was 22:1. The pressure and temperature at the bottom of the divided wall column were 80 mbar and 200° C., respectively.

A distillation in the second divided wall column according to an embodiment of the invention as shown in FIG. 1 was performed. Structured packings with a specific surface area of 495 m²/m³ were used as mass exchange elements in the divided wall column. 41% by weight of the liquid from the rectifying zone 42 was introduced to the pre-fractionating zone 41 and 59% by weight of the liquid from the rectifying zone 42 to the main fractionating zone 43. The rectifying zone 42 had 5 theoretical stages and the stripping zone 44 had 25 theoretical stages. The pre-fractionation zone 41 had 26 theoretical stages above and 14 theoretical stages below the feeding point of stream 10 into the pre-fractionation zone 41. The main fractionation zone 43 had 16 theoretical stages above and 24 theoretical stages below the withdrawal point of the side-draw product stream 34 from the main fractionating zone 43. The overhead pressure was 75 mbar. The reflux ratio at the withdrawal point of the overhead product stream 33 was 50:1. The pressure and temperature at the bottom of the divided wall column were 90 mbar and 188° C., respectively.

4000 kg/h of the reaction product stream 1 composed of 84.43% by weight of p-phenylenediamine, 5.28% by weight of o-phenylenediamine, 4.79% by weight of 2-aminobiphenyl, 3.40% by weight of diphenylamine, 0.80% by weight of aniline, 0.53% by weight of biphenyl and 0.77% by weight of tars, combined with stream 46 and was continuously fed to the pre-fractionation zone 19 of the first divided wall column through feed stream 2. The overhead product stream 10 of the first divided wall column was fed to the pre-fractionation zone 41 of the second divided wall column for further purification and the bottoms product stream 17 of the first divided wall column was removed, The liquid side-draw product stream 11 enriched in p-phenylenediamine was subjected subsequently to the suspension-based melt crystallization system 23 to obtain substantially pure p-phenylenediamine product having a purity of at least 99.99% by weight of p-phenylenediamine through stream 24. The total energy consumption was as low as 3.0 MW for the two divided wall columns. The compositions of different streams were tabulated in the following table.

| | | First divided wall column | | | |
|---|---|---|---|---|---|
| | | | | | Liquid |
| | | | Overhead | Bottoms | side-draw |
| | | Feed | product | product | product |
| | | stream | stream | stream | stream |
| P-phenylenediamine | wt. % | 88.18 | 55.23 | 15.73 | 98.65 |
| O-phenylenediamine | wt. % | 4.03 | 36.80 | 0 | 0.42 |
| 2-aminobiphenyl | wt. % | 3.76 | 0 | 41.44 | 0.60 |
| Diphenylamine | wt. % | 2.59 | 0 | 29.42 | 0.33 |
| Aniline | wt. % | 0.55 | 4.39 | 0 | 0 |
| Biphenyl | wt. % | 0.36 | 3.58 | 0 | 0 |
| Tars | wt. % | 0.53 | 0 | 13.41 | 0 |

| | | Second divided wall column | | | |
|---|---|---|---|---|---|
| | | | | | Liquid |
| | | | Overhead | Bottoms | side-draw |
| | | Feed | product | product | product |
| | | stream | stream | stream | stream |
| P-phenylenediamine | wt. % | 55.23 | 0 | 98.28 | 0.13 |
| O-phenylenediamine | wt. % | 36.80 | 22.71 | 1.42 | 99.62 |
| 2-aminobiphenyl | wt. % | 0 | 0 | 0 | 0 |
| Diphenylamine | wt. % | 0 | 0 | 0 | 0 |
| Aniline | wt. % | 4.39 | 40.36 | 0 | 0 |
| Biphenyl | wt. % | 3.58 | 36.93 | 0 | 0.26 |
| Tars | wt. % | 0 | 0 | 0.30 | 0 |

| | | Suspension-based melt crystallization | | |
|---|---|---|---|---|
| | | Feed | Product | Purge |
| | | stream | stream | stream |
| P-phenylenediamine | wt. % | 98.65 | 99.99 | 95.78 |
| O-phenylenediamine | wt. % | 0.42 | 0 | 1.32 |
| 2-aminobiphenyl | wt. % | 0.60 | 0 | 1.88 |
| Diphenylamine | wt. % | 0.33 | 0 | 1.02 |
| Aniline | wt. % | 0 | 0 | 0 |
| Bipbenyl | wt. % | 0 | 0 | 0 |
| Tars | wt. % | 0 | 0 | 0 |

### Comparative Example 2

A distillation in the aforementioned three-column distillation sequence was performed to obtain the concentrated p-phenylenediamine product having a purity of 98.65% by weight of p-phenylenediamine in the third distillation column and the highly pure o-phenylenediamine product having a purity of 99.61% by weight of o-phenylenediamine in the second distillation column, respectively. The concentrated p-phenylenediamine product from the third distillation column was subjected subsequently to a suspension-based melt crystallization system to obtain substantially pure p-phenylenediamine. The total amount of structured packings installed in the three distillation c olunms were similar as those for the two divided wall columns described in Example 2. The t otal energy consumption for the three distillation columns was 4.7 MW.

As described in the above examples according to the present invention, the total energy consumption for the two divided wall columns of the present invention is about 35% less than that for the comparative three-column distillation sequence. The first divided wll column followed by one stage suspension-based melt crystallization can prepare substantially pure p-phenylenediamine with a purity of at least 99.99% from a mixture containing o-phenylenediamine, aminobiphenyl, diphenylamine, aniline, biphenyl and tars. Consequently, the process for purification of phenylenediamines of the present invention is industrially advantageous in terms of energy consumption and equipment investment costs.

1. A continuous process for preparing substantially pure p-phenylenediamine and highly pure o-phenylenediamine from a reaction product by means of two divided wall columns coupled with at least one stage suspension-based melt crystallization, wherein the concentrated p-phenylenediamine product stream is withdrawn from the side-draw of the first divided wall column.

2. The process of claim 1, wherein said reaction product comprises 70 to 85% by weight of p-phenylenediamine, 4 to 15% by weight of o-phenylenediamine, 0 to 15% by weight of aminobiphenyls, 0 to 8% by weight of diphenylamine, 0 to 2% by weight of aniline, 0 to 2% by weight of biphenyl and 0 to 2% by weight of tars.

3. The process of claim 1, wherein the divided wall column comprises:
a divided wall provided vertically inside the column shell, defining a divided wall section between an upper undivided section as a rectifying zone and a lower undivided section as a stripping zone,
a divided wall section located between the rectifying zone and the stripping zone having a vertical dividing wall dividing the inner space of the divided wall section into a pre-fractionation zone at one side of the divided wall and a main fractionation zone at the other side of the divided wall, and
an inlet for the feed stream in the pre-fractionation zone, a side-draw outlet for the concentrated p-phenylenediamine product stream in the main fractionation zone, an overhead product stream drawn off from the rectifying zone, and a bottoms product stream removed from the stripping zone.

4. The process of claim 1, wherein said reaction product stream is continuously introduced to the inlet of the pre-fractionation zone of the first divided wall column.

5. The process of claim 1, wherein the concentrated p-phenylenediamine product stream withdrawn from the side-draw of the first divided wall column is subjected subsequently to a further purification step comprising at least one stage suspension-based melt crystallization.

6. The process of claim 5, wherein said suspension-based melt crystallization system consists of at least one scraped surface drum crystallizer and at least one wash column.

7. The process of claim 5, wherein substantially pure p-phenylenediamine having a purity of at least 99.99% by weight is obtained from said suspension-based melt crystallization.

8. The process of claim 5, wherein the purge stream removed from said suspension-based melt crystallization is recycled and combines with said reaction product stream.

9. The process of claim 1, wherein the overhead product stream from the first divided wall column is fed to the inlet of the pre-fractionation zone of the second divided wall column.

10. The process of claim 1, wherein highly pure o-phenylenediamine product stream having a purity of at least 99.5% by weight of o-phenylenediamine is obtained as the side-draw from the main fractionation zone of the second divided wall column.

11. The process of claim 1, wherein the bottoms product stream removed from the second divided wall column is recycled and combines with said reaction product stream.

12. The process of claim 1, wherein a pressure at the top of each divided wall column is in the ranges of 20 to 120 mbar.

13. The process of claim 1, wherein a pressure at the bottom of each divided wall column is the ranges of 40 to 130 mbar.

14. The process of claim 1, wherein the mass transfer elements installed in each divided wall column are selected from the group consisting of random packings, structured packings and any combinations thereof.
